(19) United States
(12) Patent Application Publication
(10) Pub. No.: US 2021/0405018 A1
(43) Pub. Date: Dec. 30, 2021

(54) APPARATUS AND TEST METHOD FOR TREATMENT FLUID SELECTION

(71) Applicant: Alchemy Sciences, Inc., Houston, TX (US)

(72) Inventors: Deepak S. Monteiro, Houston, TX (US); Luchao Jin, Houston, TX (US); James P. Russum, Sugar Land, TX (US); Erick Acosta, Sugar Land, TX (US)

(21) Appl. No.: 17/350,139

(22) Filed: Jun. 17, 2021

Related U.S. Application Data
(60) Provisional application No. 63/044,794, filed on Jun. 26, 2020.

(51) Int. Cl. G01N 33/28; G01N 21/59; G01N 21/55; G01N 21/25; G01N 1/38

(52) U.S. Cl. CPC G01N 33/2823; G01N 21/59; G01N 1/38; G01N 21/25; G01N 21/55

(57) ABSTRACT

A crude oil displacement method includes partially filling a vessel with treatment fluid and positioning an oil-saturated sample within the treatment fluid. The method also includes adding a solvent system to the vessel above the treatment fluid. In addition, the method includes moving oil from the oil-saturated sample into the solvent system. The method also includes measuring one or more of R, G, B, L, a, b, L, C, H, H, S, B, reflectance, transmittance and wavelength of the displaced oil using a color device. In addition, the method includes determining the amount of displaced oil using the one or more of R, G, B, L, a, b, L, C, H, H, S, B, reflectance, transmittance and wavelength of the displaced oil.

### CROSS-REFERENCE TO RELATED APPLICATIONS

This application is a non-provisional application which claims priority from U.S. provisional application No. 63/044,794, filed Jun. 26, 2020, which is incorporated by reference herein in its entirety.

### BACKGROUND

### Field

The disclosure relates generally to the field of treatment fluid selection in a subterranean formation during hydrocarbon recovery. More specifically the disclosure relates to methods for selecting surfactants and/or additives used in treatment fluids to improve hydrocarbon recovery.

### Background Art

Chemical additives are added to improve oil recovery from fractured conventional and unconventional reservoirs. Fractured reservoirs include a fracture system comprising both of natural fracture and hydraulic fracture and a matrix system refers to the reservoir that stores oil and consists of a network of pores. Natural fracture systems are developed in conventional and unconventional reservoirs that provide the path of fluid transport. Hydraulic fracturing is also performed to connect wellbore and formation in low permeability reservoirs and mitigate near wellbore damage. Oil recovery from fractured reservoirs compares to other reservoirs because of the limited fluid transportation from matrix into the fracture system. Traditionally, a treatment fluid including additives is introduced to underground through water injection or hydraulic fracturing to improve the oil displacement. The primary mechanisms by which the treatment fluid results in oil recovery of fractured reservoir are interfacial tension reduction and wettability alteration assisting the process of spontaneous imbibition. The spontaneous imbibition process is dominated by the capillary pressure. The reduction of interfacial tension reduces capillary entry process and increases the mobility of oil under viscous displacement. Alternating wettability to water-wet changes the capillary pressure to be favorable for treatment fluid accessing the matrix and discharging oil into the fracture network. The treatment fluid also improves the oil transport in fracture system to the wellbore that results in higher oil recovery.

### SUMMARY

A crude oil displacement method is disclosed. The method includes partially filling a vessel with treatment fluid and positioning an oil-saturated sample within the treatment fluid. The method also includes adding a solvent system to the vessel above the treatment fluid. In addition, the method includes moving oil from the oil-saturated sample into the solvent system. The method also includes measuring one or more of R, G, B, L, a, b, L, C, H, H, S, B, reflectance, transmittance and wavelength of the displaced oil using a color device. In addition, the method includes determining the amount of displaced oil using the one or more of R, G, B, L, a, b, L, C, H, H, S, B, reflectance, transmittance and wavelength of the displaced oil.

A crude oil displacement method is disclosed. The method includes partially filling a vessel with a treatment fluid and positioning an oil-saturated sample within the treatment fluid. The method also includes adding a solvent system to the vessel above the treatment fluid and displacing oil from the oil-saturated sample. The oil is moved into the solvent system. In addition, the method includes measuring a color difference between the displaced oil and a standard color to determine the amount of displaced oil.

A crude oil displacement method is disclosed. The method includes partially filling a vessel with treatment fluid and positioning an oil-saturated sample within the treatment fluid. The method also includes adding a solvent system to the vessel above the treatment fluid and moving oil from the oil-saturated sample into the solvent system. The method further includes photographing the displaced oil and using a color picker application or color detections software to obtain R, G, B, L, a, b, L, C, H, H, S, B, reflectance, transmittance or wavelength values from the photograph. In addition, the method includes comparing the values of R, G, B, L, a, b, L, C, H, H, S, B, reflectance, transmittance or wavelength against a calibration curve or correlation to obtain displaced oil concentration. A crude oil displacement measurement apparatus is disclosed. The apparatus includes a base, the base including a plurality of rows of vial receptors, the vial receptors adapted to receive vials. The apparatus also includes a dispenser robotic arm, the dispenser robotic arm extending across the base parallel with the row of vial receptors, the dispenser robotic arm adapted to move transversely across the rows of vial receptors. The apparatus further includes a plurality of automated dispensers positioned on the dispenser robotic arm and a placement robotic arm, the placement robotic arm adapted to move transversely across the rows of vial receptors and along the rows of vial receptors. The placement robotic arm includes an elevating arm, a rotating arm extending at an angle from the elevating arm, and a gripper, the gripper mechanically connected to the rotating arm, the gripper adapted to grip a vial. The apparatus includes a color device and a color device robotic arm, the color device robotic arm mechanically connected to the color device, the color device robotic arm adapted to move across a row of vial receptors.

### BRIEF DESCRIPTION OF THE DRAWINGS

The present disclosure is best understood from the following detailed description when read with the accompanying figures. It is emphasized that, in accordance with the standard practice in the industry, various features are not drawn to scale. In fact, the dimensions of the various features may be arbitrarily reduced for clarity of discussion.

FIG. 1 is a graphical depiction of a test method to detect oil recovery via spontaneous imbibition consistent with at least one embodiment of the present disclosure.

FIG. 2 is a depiction of a high throughput apparatus for determination of hydrocarbon recovery during spontaneous imbibition consistent with at least one embodiment of the present disclosure.

FIG. 3 is a graph of P values for Example 1.

FIG. 4 is a graph of a prediction profiler output based on the P values shown in FIG. 3.

### DETAILED DESCRIPTION

The following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

This disclosure is not limited to the embodiments, versions, or examples described, which are included to enable a person having ordinary skill in the art to make and use the disclosed subject matter when the information contained herein is combined with existing information and technology.

Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations. For example, if the detailed description recites a range of from 1 to 5, that range includes all iterative ranges within that range including, for instance, 1.3-2.7 or 4.9-4.95.

The present disclosure relates to methods and an apparatus for measuring oil recovery from the process of spontaneous imbibition. Oil recovery methods most often include the use of treatment fluids. Treatment fluids are most often water-based and include a number of additives. These additives typically include, but are not limited to, acids, biocides, emulsion breakers, corrosion inhibitors, hydrate inhibitors, friction reducers, gels, iron control chemicals, oxygen scavengers, surfactants, wettability additives, salts, polymers and scale inhibitors. The treatment fluid may be dosed into a brine prior to treatment. During oil recovery operations, the treatment fluid in combination with the hydrocarbon may flow from the matrix to the fracture network. The treatment fluid and hydrocarbons may then flow from the fracture network to the wellbore.

The additive in the treatment fluid may act to increase oil recovery from oil/gas reservoirs, such as by enhancing water imbibition into the matrix and aiding oil flow from the fracture network to the wellbore. Selection of an additive for the treatment fluid may be determined by a number of factors, including, but not limited to wettability, interfacial surface tension, viscosity reduction, density reduction, ability to mobilize linear hydrocarbons, ability to decompose asphaltenes and bitumen, ability to emulsify, ability to breakdown carbon chains to smaller and the ability to accelerate oil recovery and decrease the time necessary to measure initial oil recovery. Improper selection of an additive may result in inefficient or ineffective oil recovery.

Brine refers to either produced brine, fresh brine or combinations thereof. The brine salinity and ions may vary.

Processes in which such treatment fluids may be used may include, but are not limited to, hydraulic fracturing treatments, enhanced oil recovery treatments (including, for instance, water flooding treatments and polymer flooding treatments), and acidizing treatments. In certain embodiments, the low permeability reservoir may be contacted by the treatment fluid, such as, for instance, introduction into a wellbore that penetrates the low permeability reservoir.

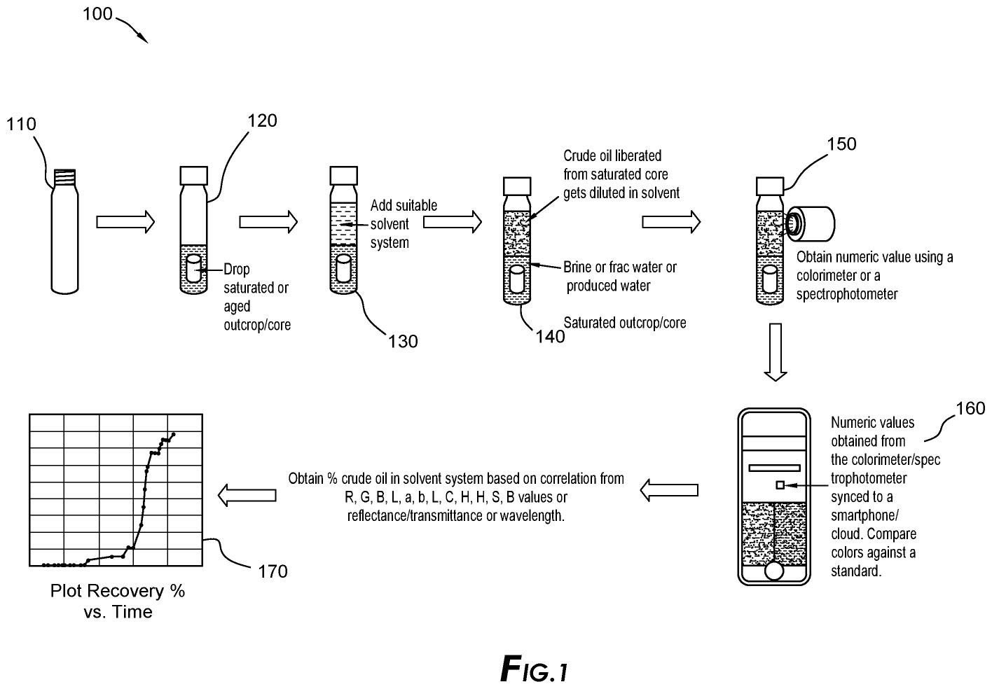

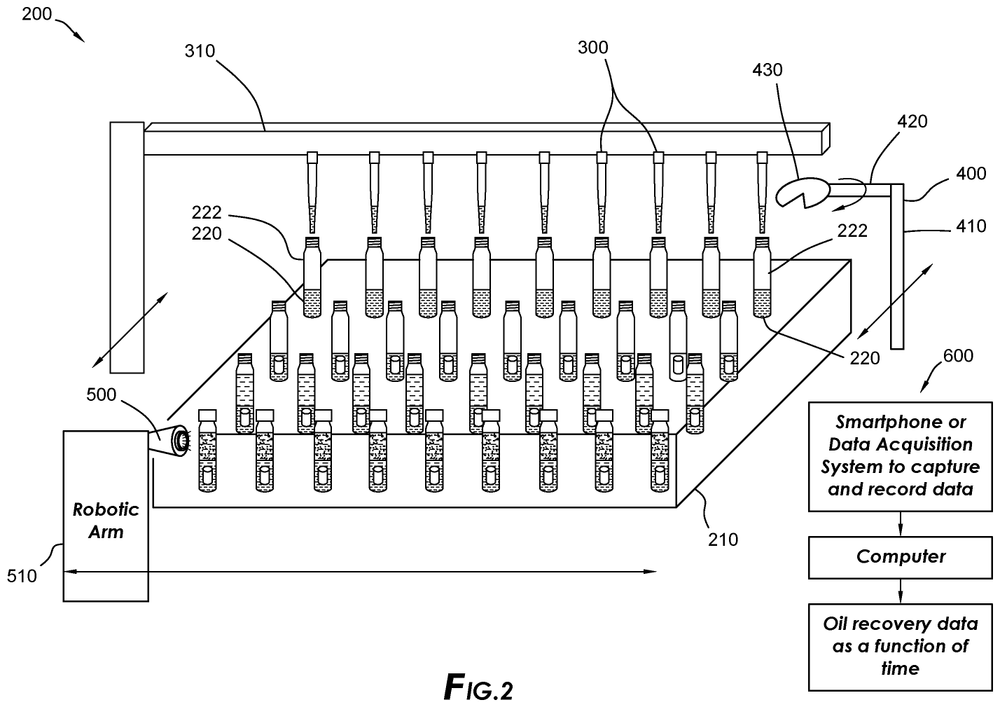

The present disclosure includes tests and test apparatuses that may be performed to select additives for treatment fluids. One such test method is depicted in FIG. 1. An exemplary apparatus for using the test method depicted in FIG. 1 is depicted in FIG. 2.

FIG. 1 shows crude oil displacement measurement method 100. A vial or other vessel is partially filled with treatment fluids and additives in step 110. An oil-saturated core is positioned within the treatment fluid in the vial in step 120. The saturated cored plug is a sample of the reservoir rock varying from discs to cylindrical cores that are smaller or the same size as typically used in Amott cell tests. In alternate embodiments, mud cuttings are used to evaluate. The saturated core may include such substances as calcite, sandstone, quartz, clay, or combinations thereof. In yet other embodiments, outcrops may be used as an analog of reservoir rock. To form the saturated core plug, a core plug is saturated with crude oil.

A solvent system consisting of a single solvent or solvent mixtures is then added, such as by pipette into the vial above the brine in step 130. The solvent system used may include, but not be limited to, Toluene, Xylene, Benzene, Pentane, Hexane, Heptane, Methanol, Isopropanol, 2 Ethyl-Hexanol, Ethylene Glycol, Ethylene Glycol Monobutyl Ether, Glycerol, Terpenes, Water, and combinations thereof.

Water imbibes into the saturated core plug and moves crude oil from the saturated core plug, and the oil that has been moved is diluted in the solvent system in step 140. Without being bound by theory, the process of moving oil from the saturated core plug into the solvent system may include multiple steps. For example, oil may be displaced from the oil-saturated sample. This displaced oil may be detached from the sample surface, and then migrated and dissolved into the solvent system. “Migration” may include the movement of the oil after detachment and prior to the dissolution in the solvent system.

The moved oil is measured using a color device such as a colorimeter or a spectrophotometer in step 150. The colorimeter, spectrophotometer or a color detector returns values for R, G, B, L, a, b, L, C, H, H, S, B, reflectance, transmittance and wavelength values in step 150. Alternately, color detection software can be used to obtain R, G, B, L, a, b, L, C, H, H, S, B, reflectance, transmittance, wavelength values to obtain oil recovery data based on measurement by the colorimeter or spectrophotometer. These values may be compared against a standard in step 160. The calibration curve may be used to obtain the concentration of displaced oil in the solvent system in step 170, and therefore the concentration of displaced oil. In another embodiment, the color difference between the displaced oil and a standard color (ex: maroon, dark red, brown, firebrick, crimson etc.) may be used to determine the amount of displaced oil.

Steps 150, 160, and 170 may be repeated over time to obtain oil displaced as a function of time. By repeating displacement measurement method 100 with different treatment fluids, the amount of displaced oil may be compared for the different treatment fluids. In another embodiment, photographs may be captured of the displaced oil in the solvent system at different intervals. A color picker application or a color detection software may be used to obtain numerical values of R, G, B, L, a, b, L, C, H, H, S, B, reflectance, transmittance and wavelength values from the photographs. These values may be compared against a calibration curve or a correlation to obtain the displaced oil concentration and displaced oil concentration over time.

FIG. 2 depicts crude oil displacement measurement apparatus 200. Crude oil displacement measurement apparatus 200 includes base 210 on which a plurality of rows of vial receptors 220 are positioned. Vial receptors 220 may be depression or holes in base 210 adapted to receive vials 222 or other containers. Crude oil displacement measurement apparatus 200 further includes automated dispensers 300 adapted to dispense the brine and the solvent system. Automated dispensers 300 may be positioned on dispenser robotic arm 310 that extends across base 210 parallel with a row of vial receptors 220. Dispenser robotic arm 310 is adapted to move transversely across rows of vial receptors 220 such that automated dispensers may inject fluid into the rows of vials 222. Transverse movement of dispenser robotic arm may be accomplished by a geared track or other mechanism. In certain embodiments, crude oil displacement measurement apparatus 200 includes placement robotic arm 400. Placement robotic arm 400 may place the saturated cores in vials 222, to move vials 222 and to cap vials 222. Placement robotic arm 400 may traverse both transversely across rows of vial receptors 220 as well as along rows of vial receptors 220 on a geared track or other mechanism. Placement robotic arm may include elevating arm 410 mechanically coupled to rotating arm 420. Rotating arm 420 may extend at an angle, such as a perpendicular angle to elevating arm 410. Rotating arm 420 may terminate in gripper 430, adapted to grip vials 222 and place or remove caps on vials 222. Gripper 430 may rotate in a plane parallel to the surface of base 210. As described above, R, G, B, L, a, b, L, C, H, H, S, B, reflectance, transmittance and wavelength values of the oil in the solvent system may be measured by color device 500, such as a spectrophotometer, colorimeter, or color detector. Color device 500 may be mounted on color device robotic arm 510 to obtain data from vials 222. Color device robotic arm 510 may move across a row of vial receptors 220 to measure R, G, B, L, a, b, L, C, H, H, S, B, reflectance, transmittance and wavelength values such as on a geared track or other mechanism. These measured values from color device 500 may be data acquisition and analysis system 600, which may be a smartphone, other computing device, or a combination. Data acquisition and analysis system 600 may also determine oil recovery data through instructions digitally stored in a non-transitory media.

In another embodiment of crude oil displacement measurement apparatus 200, vials 222 may be replaced by visual cells adapted to withstand elevated pressures and temperatures. A positive displacement pressure may be applied to push oil out of the high pressure and temperature cell using a relief valve or a regulator and the oil coming out the pressure cell is diluted in a solvent system. The pushed oil is then measured using color device 500 to calculate the concentration of crude oil in the solvent system. The oil recovered is then calculated from the concentration of crude oil in the solvent system.

In another embodiment of the crude oil displacement measurement apparatus 200, Hassler core holders may be used instead of vials 222 and the oil coming out of the core holder may be measured using color device 500 to determine the amount of oil displaced with elevated pressure and temperature.

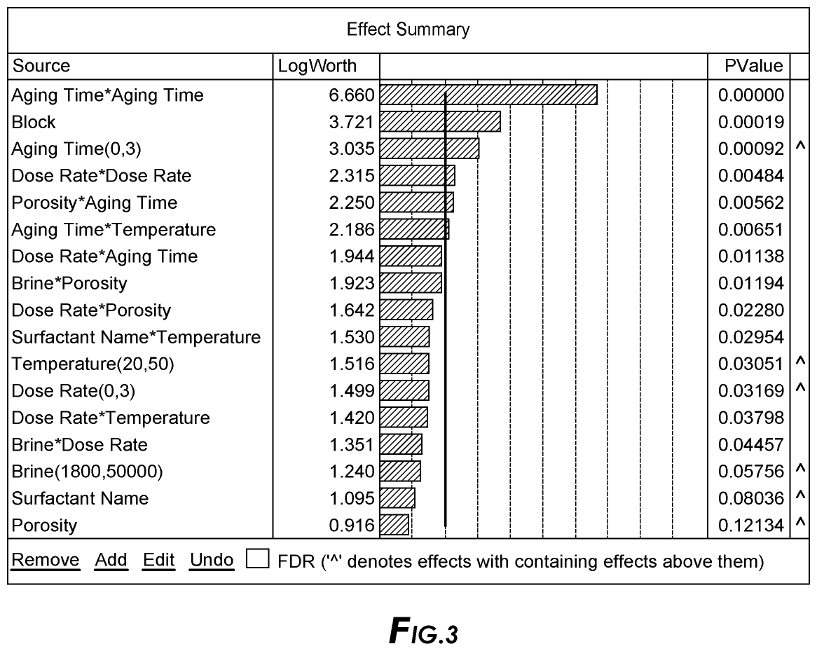

In certain embodiments, once color data is obtained and oil recovery is estimated, screening and sensitivity analyses are conducted on recovery based on identifying and changing a number of factors. Examples of factors are shown in FIG. 3. Sensitivity analysis may allow for the convergence on an optimal set of factors to improve product efficacy and identify the performance envelope related to the treatment fluid. This set of factors may assist in determining field applicability based on the performance envelope.

### Example

The crude oil measurement displacement method was used to efficacy as well as determine the sensitivity of different factors on crude oil recovery. Edwards limestone core plugs were used for this study and the cores were saturated with crude oil from the Eagle Ford shale prior to aging. Aging of the core plugs was conducted at 80° C. in an oven based on the time specified as Aging Time. The core plugs were then placed into a brine/surfactant solution after saturation and aging and the oil displaced was measured using a spectrophotometer.

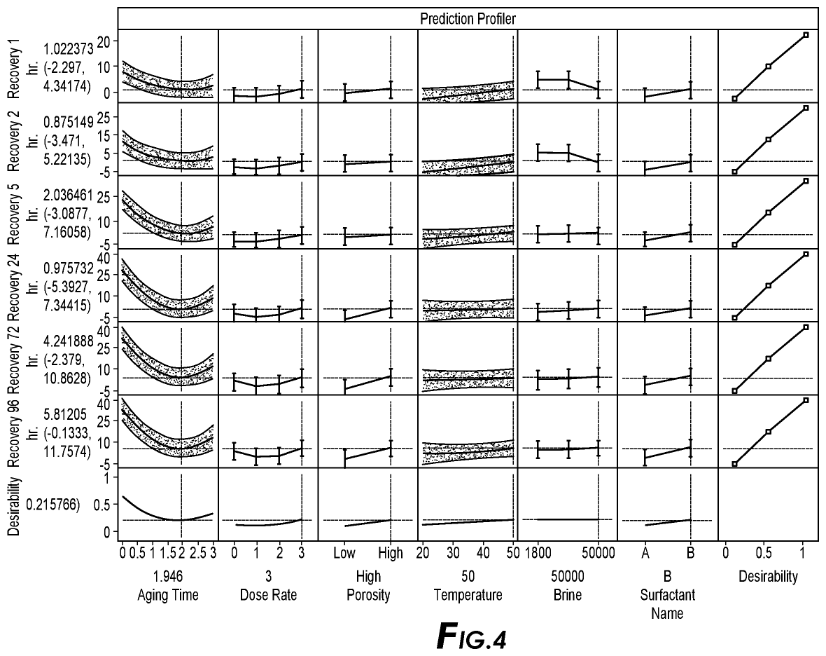

The factors studied were Surfactant name (A), Brine salinity (1800 ppm:frac. water, 5000 ppm:90/10 mix & 50000 ppm:prod. water), Porosity (Low Porosity:14.09-15.55 & High Porosity:24.8-26.17)—estimated from mass and volume measurements, Aging time (0, 1, & 2 weeks), Test Temperature (20, 35, & 50° C.), Dose Rate (0, 1, 2, & 3 gpt). The response was the crude oil recovery at different intervals of time (1, 2, 5, 24, 48, & 72 hr.). FIG. 3 shows effect summary and FIG. 4. shows the prediction profiler from 47 experiments. The effect summary shows the different factors as well as the interactions between different factors which effect the response. P values are indicated in FIG. 3, A P-value below 0.05 indicates that the data is statistically significant. The prediction profiler is shown in FIG. 4. using the effect summary in FIG. 3. As can be determined from the data in FIG. 4:
Higher recovery is obtained from high porosity core plugs compared to low porosity core plugs.
Aging time has an negative impact on oil recovery. An aging time of −2 weeks appears to be the optimum aging time for the Edwards limestone core plugs and the Eagle Ford shale oil.
An increase in test temperature favors oil recovery.
B outperforms A under the conditions tested.
3 gpt is the optimum dose rate for either surfactant and at this dose rate better recovery is obtained compared to the blank for aged core plugs.

The foregoing outlines features of several embodiments so that a person of ordinary skill in the art may better understand the aspects of the present disclosure. Such features may be replaced by any one of numerous equivalent alternatives, only some of which are disclosed herein. One of ordinary skill in the art should appreciate that they may readily use the present disclosure as a basis for designing or modifying other processes and structures for carrying out the same purposes and/or achieving the same advantages of the embodiments introduced herein. One of ordinary skill in the art should also realize that such equivalent constructions do not depart from the spirit and scope of the present disclosure and that they may make various changes, substitutions, and alterations herein without departing from the spirit and scope of the present disclosure.

What is claimed is:

1. A crude oil displacement method comprising:
a) partially filling a vessel with treatment fluid;
b) positioning an oil-saturated sample within the treatment fluid;
c) adding a solvent system to the vessel above the treatment fluid;
d) moving oil from the oil-saturated sample=the solvent system;
e) measuring one or more of R, G, B, L, a, b, L, C, H, H, S, B, reflectance, transmittance and wavelength of the displaced oil using a color device; and
f) determining the amount of displaced oil using the one or more of R, G, B, L, a, b, L, C, H, H, S, B, reflectance, transmittance and wavelength of the displaced oil.

2. The method of claim 1 wherein the step of determining the amount of displaced oil includes comparing the one or more of R, G, B, L, a, b, L, C, H, H, S, B, reflectance, transmittance and wavelength of the displaced oil against a standard.

3. The method of claim 2, wherein the step of determining the amount of displaced oil includes combining the reflectance or transmittance of the displaced oil with the wavelength to obtain a calibration curve.

4. The method of claim 3, wherein the calibration curve is used to determine the amount of displaced oil.

5. The method of claim 1, wherein steps a) through f) are repeated at different times, the method further comprising determining the amount of displaced oil over time.

6. The method of claim 1, wherein the solvent system comprises Toluene, Xylene, Benzene, Pentane, Hexane, Heptane, Methanol, Isopropanol, 2 Ethyl-Hexanol, Ethylene Glycol, Ethylene Glycol Monobutyl Ether, Glycerol, Terpenes, Water, and combinations thereof.

7. A crude oil displacement method comprising:
partially filling a vessel with a treatment fluid;
positioning an oil-saturated sample within the treatment fluid;
adding a solvent system to the vessel above the treatment fluid;
moving oil from the oil-saturated sample into the solvent system; and
measuring a color difference between the displaced oil and a standard color to determine the amount of displaced oil.

8. A crude oil displacement method comprising:
a) partially filling a vessel with treatment fluid;
b) positioning an oil-saturated sample within the treatment fluid;
c) adding a solvent system to the vessel above the treatment fluid;
d) moving oil from the oil-saturated sample into the solvent system;
e) photographing the displaced oil;
using a color picker application or color detections software to obtain R, G, B, L, a, b, L, C, H, H, S, B, reflectance, transmittance or wavelength values from the photograph; and
comparing the values of R, G, B, L, a, b, L, C, H, H, S, B, reflectance, transmittance or wavelength against a calibration curve or correlation to obtain displaced oil concentration.

9. A crude oil displacement measurement apparatus comprising:
a base, the base including a plurality of rows of vial receptors, the vial receptors adapted to receive vials;
a dispenser robotic arm, the dispenser robotic arm extending across the base parallel with the row of vial receptors, the dispenser robotic arm adapted to move transversely across the rows of vial receptors;
a plurality of automated dispensers positioned on the dispenser robotic arm;
a placement robotic arm, the placement robotic arm adapted to move transversely across the rows of vial receptors and along the rows of vial receptors, the placement robotic arm comprising:
an elevating arm;
a rotating arm extending at an angle from the elevating arm;
a gripper, the gripper mechanically connected to the rotating arm, the gripper adapted to grip a vial;
a color device; and
a color device robotic arm, the color device robotic arm mechanically connected to the color device, the color device robotic arm adapted to move across a row of vial receptors.

10. The crude oil displacement measurement apparatus of claim 8, wherein the color device is a spectrophotometer, a colorimeter, or a color detector.

11. The crude oil displacement measurement apparatus of claim 8, wherein the color device is in data communication with a data acquisition and analysis system.
